(19) United States
(12) Patent Application Publication
(10) Pub. No.: US 2023/0363633 A1
(43) Pub. Date: Nov. 16, 2023

(54) VIDEO LARYNGOSCOPE SYSTEM AND METHOD FOR QUANTITATIVELY ASSESSMENT TRACHEA

(71) Applicant: Covidien LP, Mansfield, MA (US)

(72) Inventors: Mingxia SUN, Carlsbad, CA (US); Chunlang HONG, Carlsbad, CA (US); Jianfeng GU, Shanghai (CN); Junhua SONG, Shanghai (CN)

(21) Appl. No.: 18/247,016

(22) PCT Filed: Oct. 22, 2020
(86) PCT No.: PCT/CN2020/122676

§ 371 Date: Mar. 28, 2023

(51) Int. Cl. A61B 1/267; A61B 1/00; G06T 7/187; G06T 7/13

(52) U.S. Cl. CPC A61B 1/2673; A61B 1/000094; A61B 1/0005; G06T 7/187; G06T 7/13; A61B 2090/061

(57) ABSTRACT

The present disclosure provides a video laryngoscope system comprising an image acquisition device configured to capture images of glottis and trachea of a subject, a memory configured to store one or more series of instructions, one or more processor configured execute the series of computer instructions stored in the memory. When the instructions are executed by the processor, the video laryngoscope system performs the following steps: receiving the images of the glottis and the trachea captured by the image acquisition device, analyzing the received images to identify a tracheal structure, and quantitatively assessing the trachea based on the identified tracheal structure, to determine at least one attribute of the trachea. The present disclosure further provides a method for quantitatively assessing a trachea.

### FIELD OF THE INVENTION

The present disclosure relates in general to medical devices, and in more particular, to a video laryngoscope system and method for quantitatively assessing trachea.

### BACKGROUND OF THE INVENTION

Intubation is critical to the care of patients who are undergoing anesthesia during surgery, or who appear in trauma centers for acute myocardial infarction, respiratory distress or removal of foreign bodies. It is thought to be important to select the appropriate size of endotracheal tube (ETT) to prevent ETT-induced complications, such as airway edema. For example, an overinflated cuff or excessively large ETT relative to tracheal size may induce tracheal mucosal ischemia or hoarseness. To the contrary, an uninflated/underinflated cuff or small ETT relative to tracheal size may induce the leaking of respiratory gases. This concern is also critical in children due to the smaller caliber of the pediatric airway and the potentially lifelong impact of airway injury.

Therefore, there is a requirement for quantitatively assessing trachea so as to determine the correct size of the ETT for any individual subject.

### SUMMARY OF THE INVENTION

According to one aspect of the disclosure, a video laryngoscope system is provided, and the system comprises: an image acquisition device configured to capture images of glottis and trachea of a subject, a memory configured to store one or more series of instructions, one or more processor configured execute the series of computer instructions stored in the memory. When the instructions are executed by the processor, the video laryngoscope system performs the following steps: receiving the images of the glottis and the trachea captured by the image acquisition device, analyzing the received images to identify a tracheal structure, and quantitatively assessing the trachea based on the identified tracheal structure, to determine at least one attribute of the trachea.

In some embodiments of the present disclosure, an image segmentation algorithm is applied to the captured images to identify the tracheal structure.

In some embodiments of the present disclosure, the image segmentation algorithm includes at least one of region growing algorithms, segmentation algorithms based on edge detection, segmentation algorithms based on neural network and segmentation algorithms based on machine learning.

In some embodiments of the present disclosure, a representation of the identified tracheal structure is superimposed on the received image and displayed on a display of the video laryngoscope system.

In some embodiments of the present disclosure, at least one attribute of the trachea comprises at least one of a diameter of the trachea, a radius of the trachea, a perimeter of the trachea, an area of the trachea.

In some embodiments of the present disclosure, a representation of the at least one attribute of the trachea is displayed on a display of the video laryngoscope system.

In some embodiments of the present disclosure, the representation of the at least one attribute of the trachea is superimposed on the received image.

In some embodiments of the present disclosure, the representation of the at least one attribute of the trachea comprises graphical representation and numerical representation of the attribute of the trachea.

In some embodiments of the present disclosure, the at least one attribute of the trachea is output by a speaker

In some embodiments of the present disclosure, the image acquisition device has predetermined magnification and object distance, and is positioned such that the glottis is in focus.

In some embodiments of the present disclosure, a reference object with known size is positioned near the glottis and is captured by the image acquisition device.

In some embodiments of the present disclosure, the system further comprises a distance measuring device configured to measure the distance between a lens of the image acquisition device and the tracheal structure.

According to another aspect of the disclosure, a method for quantitatively assessing a trachea is provided. The method comprises: receiving images of glottis and trachea of a subject captured by an image acquisition device of a video laryngoscope system, analyzing the received images to identify a tracheal structure, and quantitatively assessing the trachea based on the identified tracheal structure, to determine at least one attribute of the trachea.

Further scope of applicability of the present disclosure will become apparent from the detailed description given hereinafter. However, it should be understood that the detailed description and specific examples, while indicating preferred embodiments of the present disclosure, are given by way of illustration only, since various changes and modifications within the spirit and scope of the present disclosure will become apparent to those skilled in the art from the following detailed description.

### BRIEF DESCRIPTION OF THE DRAWINGS

The above and other aspects and advantages of the present disclosure will become apparent from the following detailed description of exemplary embodiments taken in conjunction with the accompanying drawings which illustrate, by way of example, the principles of the present disclosure. Note that the drawings are not necessarily drawn to scale.

FIG. 1 shows a schematic diagram illustrating a block diagram of the video laryngoscope system according to at least one embodiments of the present disclosure.

FIG. 2 shows a process flow diagram illustrating a method for quantitatively assessing a trachea according to the embodiments of the present disclosure.

FIG. 3 shows a drawing illustrating the image of the glottis and trachea captured by the image acquisition device according to at least one embodiments of the present disclosure.

FIG. 4 shows a drawing illustrating the image of FIG. 3 after image segmentation and quantitative assessment of the trachea according to at least one embodiments of the present disclosure.

### DETAILED DESCRIPTION OF THE PREFERRED EMBODIMENTS

In the following detailed description, numerous specific details are set forth to provide a thorough understanding of the described exemplary embodiments. It will be apparent, however, to one skilled in the art that the described embodiments can be practiced without some or all of these specific details. In other exemplary embodiments, well known structures or process steps have not been described in detail in order to avoid unnecessarily obscuring the concept of the present disclosure.

In the related art, tracheal diameter can generally be measured accurately by CT, but CT images are taken only for a limited number of patients. Also, it is time-consuming and uneconomical to take CT image for each patient.

Further, chest X-ray images are often taken preoperatively, and used to determine the diameter of the trachea so as to determine the ETT size. However, as tracheal diameter measured by X-ray is not always accurate.

Visualization of the patient's anatomy during intubation can help the clinician to avoid damaging or irritating the patient's oral and tracheal tissue, and avoid passing the ETT into the esophagus instead of the trachea. The clinician may use a video laryngoscope which contains a video camera oriented toward the patient, and thus he/she can obtain an indirect view of the patient's anatomy by viewing the images captured from the camera and displayed on a display screen. This technology allows the anesthetist to truly view the position of the ETT on a video screen while it is being inserted, and video laryngoscope could reduce the risks of complications and intubation failure further.

As described in detail below, embodiments of a video laryngoscope system are provided herein. In particular, embodiments of the present disclosure relate to a system for quantitatively assessing trachea based on image or video collected from the airway by an image acquisition device of the video laryngoscope system. The term “quantitative” herein means that the quantitative assessment of the trachea determines the value or number of the attributes relating to the trachea. The quantitative assessment of the patient's trachea may be used to select an appropriately-sized ETT. Therefore, it is possible to avoid the inappropriate ETT size induced complications. In particular, by allowing the operator (for example, clinician) to select the appropriate ETT promptly, it is possible to avoid the increment of the partial pressure of the volatile anesthetic in the body as well as apnea and bradycardia that may have been induced. Further, when the quantitative assessment of the patient's trachea is the tracheal diameter, it is possible to provide accurate tracheal diameter. In some embodiments of the present disclosure, the tracheal diameter information may be used to control inflation of a cuff of the ETT. That is, a desired inflation volume for a cuff may be selected according to the determined tracheal diameter.

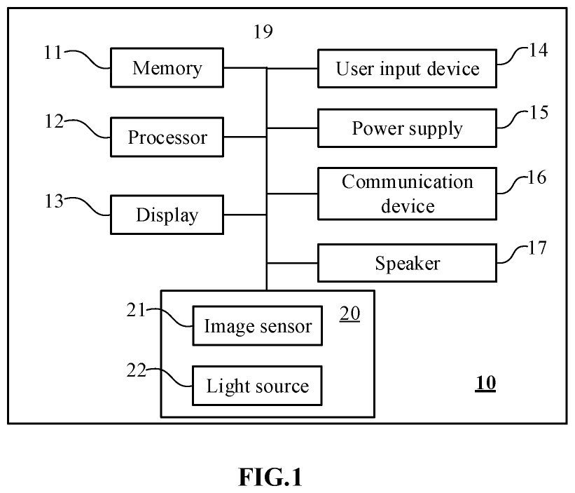

Turning now to the figures, FIG. 1 shows a block diagram of the video laryngoscope system 10 according to at least one embodiments of the present disclosure. As shown in FIG. 1, in at least one embodiments of the present disclosure, the video laryngoscope system 10 includes, for example, a memory 11, one or more processors 12, a display 13 and an image acquisition device 20. Further, the video laryngoscope system 10 may comprises an user input device 14, a power supply 15, a communication device 16 and a speaker 17. At least some of these components are coupled with each other through an internal bus 19.

The function and operation of the image acquisition device 20 of the video laryngoscope system 10 is described below. While the image acquisition device 20 may be external to the subject, it is envisioned that the image acquisition device 20 may also inserted directly into the subject's airway to capture the image of the oral or tracheal structure, prior to or concurrently with an airway device (for example, prior to the ETT), so as to capture images that may be sent to the memory 11 for storage and/or to the one or more processors 12 for further processing. In some embodiment, the image acquisition device 20 may be formed as an elongate extension or arm (e.g., metal, polymeric) housing an image sensor 21 for capturing images of the tissue of the subject and a light source 22 for illuminating the tissue of the subject. The image acquisition device 20 may also house electrical cables (not shown) that couple the image sensor 21 and the light source 22 to other components of the video laryngoscope system 10, such as the one or more processors 12, the display 13, the power source 15 and the communication device 16. The electrical cables provide power and drive signals to the image sensor 21 and light source 22 and relay data signals back to other components of the video laryngoscope system 10. In certain embodiments, these signals may be provided wirelessly in addition to or instead of being provided through electrical cables.

In use to intubate a patient, a removable and at least partially transparent blade (not shown) is slid over the image acquisition device 20 like a sleeve. The laryngoscope blade includes an internal channel or passage sized to accommodate the image acquisition device 20 and to position an image sensor 21 of the image acquisition device 20 at a suitable angle to visualize the airway. The laryngoscope blade is at least partially transparent (such as transparent at the image sensor 21, or transparent along the entire blade) to permit the image sensor 21 of the image acquisition device 20 to capture images through the laryngoscope blade. The image sensor and light source of the image acquisition device 20 facilitate the visualization of an ETT or other instrument inserted into the airway. The laryngoscope blade may be selected to an appropriate patient size and shape based on an estimate or assessment of the patient's airway, size, or condition, or according to procedure type, or operator preference.

In some embodiments of the present disclosure, instead of the blade laryngoscope, the video laryngoscope system 10 may comprises a fiber optic laryngoscope. The similar configuration can be applied to the optic fiber laryngoscope and the detailed description thereof is omitted here.

The memory 11 is configured to store one or more series of instructions, and the one or more processors 12 are configured to execute the instructions stored in the memory 11 so as to control the operation of the video laryngoscope system 10 and perform the method as disclosed in the present disclosure. For example, the one or more processors 12 may execute instructions stored in the memory 11 to send to and receive signals from the image sensor 21 and to illuminate the light source 22. The received signals include image and/or video signals to be displayed on the display 13. In the embodiments of the present disclosure, the received video signal from the image sensor 21 will be processed according to instructions stored in the memory 11 and executed by the processor 12. The memory 11 may include other instructions, code, logic, and/or algorithms that may be read and executed by the processor 12 to perform the techniques disclosed herein.

The processing of the one or more processors 12 will be described in detail later.

In addition to the video signals from the image acquisition device 20, the display 13 may also be used for display of other information, e.g., the parameters of the video laryngoscope system 10 and indication of the inputs provided by the user. Further, as discussed below, the display 13 can also displays the quantitative assessment of the trachea determined according to the embodiment of the present disclosure.

The display 13 can be integrated with the components of the video laryngoscope system 10, such as mounted on the handle of the laryngoscope that is gripped and manipulated by the operator, within the operator's natural viewing angle looking toward the patient, to enable the operator to view the display while manipulating the laryngoscope and ETT in real time. Accordingly, the user can view the integrated display to guide the ETT in the airway while also maintaining visual contact with the airway entry to assist in successful intubation.

In some embodiments of the present disclosure, a remote display or medical rack display can be adopted, and thus the display 13 can be separated from other components of the video laryngoscope system 10 and coupled with the other components via a wire or wirelessly.

The video laryngoscope system 10 may further comprises user input device 14 such as knobs, switches, keys and keypads, buttons, etc., to provide for operation and configuration of the system 10. In case that the display 13 is a touch screen, the display 13 may constitute at least part of the user input device 14.

The video laryngoscope system 10 may also include a power source 15 (e.g., an integral or removable battery or a power cord) that provides power to one or more components of the video laryngoscope system 10. Further, the video laryngoscope system 10 may also include communications device 16 to facilitate wired or wireless communication with other devices. In one embodiment, the communications device may include a transceiver that facilitates handshake communications with remote medical devices or full-screen monitors. The communications device 16 may provide the images displayed on the display 13 to additional displays in real time. Moreover, the video laryngoscope system 10 may also include speaker 17 that output audible information.

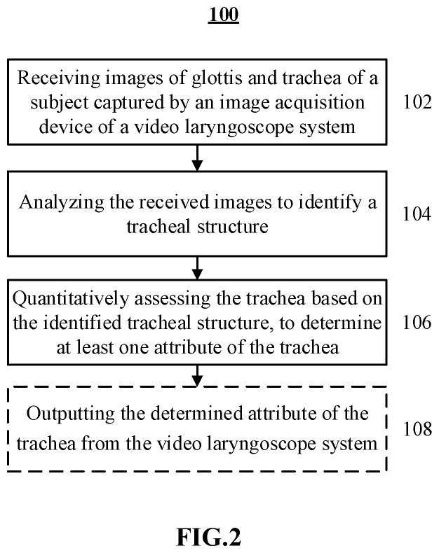

FIG. 2 is a process flow diagram illustrating a method 100 for quantitatively assessing a trachea according to the embodiments of the present disclosure. The method may be performed as an automated procedure by a system, such as the video laryngoscope system 10 of the present disclosure. For example, certain steps may be performed by the one or more processors 12, that executes stored instructions for implementing steps of the method 100. In addition, in particular embodiments, certain steps of the method 40 may be implemented by the operator.

According to a particular embodiment, at step 102, the images of glottis and tracheal structure captured by image acquisition device are received. The images of the glottis and tracheal structure are captured by the image sensor 21 of the image acquisition device 20 which is in inserted directly into the subject's airway. Then, at step 104, the received images are analyzed to identify the structure of the trachea. The analysis of the images is performed by the one or more processors 12, and the details of the process will be described latter. By analyzing the captured images, the image of the trachea contained in the captured image is extracted and the structure of the trachea can be identified from the extracted image. Then, at step 106, the trachea is quantitatively assessed based on the identified tracheal structure, to determine at least one attribute of the trachea. The at least one attribute of the trachea comprises, for example, the diameter of the trachea (airway), the perimeter of the trachea and the area of the trachea. Based on the determined diameter of the trachea, the operator can select the ETT with appropriate size. At an option step 108, the determined attribute of the trachea can be output from the video laryngoscope system 10. For example, the attribute can be displayed on the display 13 or output by a speaker 17.

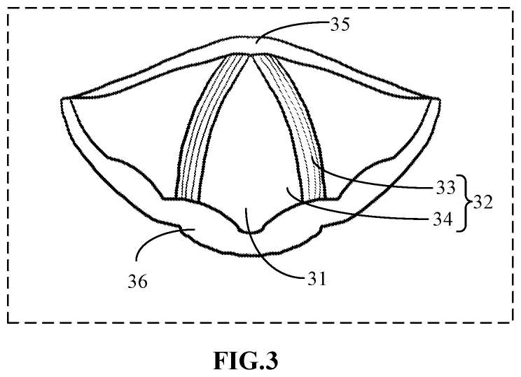

FIG. 3 shows a drawing illustrating the image of the glottis and trachea captured by the image acquisition device (i.e., image sensor). As shown in FIG. 3, the glottis 32 comprises vocal cord 33 and the glottis aperture 34 formed by the vocal cord 33 and the arytenoid cartilage 36. The trachea 31 can be seen through the glottis aperture 34. Further, the epiglottis 35 is also shown in FIG. 3.

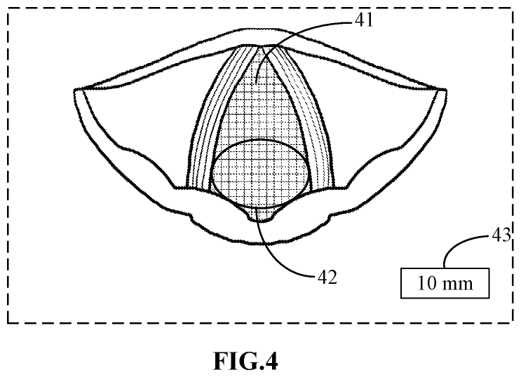

Further, the image shown in FIG. 3 is analyzed and the structure of the trachea 31 therein is identified. FIG. 4 shows a drawing illustrating the image of FIG. 3 after image segmentation. The part of the image corresponding to the trachea 31 is identified and extracted by applying, for example, image segmentation algorithm to the capture image. In the present disclosure, as shown in FIG. 4, the part of the image within the glottis aperture 34, that is, between the vocal cord 33 and the arytenoid cartilage 36, is the image of the trachea 31 and is marked by the gridding in FIG. 4. Thus, the structure of the trachea 31 can be identified from the extracted image.

Various image segmentation algorithms known in the art may be employed to segment the tracheal structure. For example, conventional image segmentation methods may be employed, e.g., region growing algorithms, edge-based segmentation algorithms. In addition, an artificial intelligence (AI) segmentation algorithm or the like may also be employed, e.g., the segmentation algorithms based on neural network or machine learning. Taking region growing algorithms as an example, with such algorithms, the pixel points with similar properties are connected and combined. In each area, one seed point is used as a growth starting point, and then the growth and combination are carried out on the pixel points in the field arranged around the seed point according to the growth rule until no pixel which can meet the growth point exists. The detailed descriptions of these algorithms are omitted here. It can be appreciated that image segmentation algorithms are not limited to the above specific examples.

In some embodiments of the present disclosure, as shown in FIG. 4, the representation 41 (e.g., gridding) of the identified tracheal structure can be superimposed on the received image from the image acquisition device 20, as shown in FIG. 4 and displayed on the display. Therefore, the operator may intuitively confirm whether the identified tracheal structure is correct. For example, if the identified tracheal structure is not correct, the operator may notice it by the misplacement of the gridding. Then, the operator may instruct the system to correct the identified tracheal structure, for example, by moving the image acquisition device 20 and acquiring a new image of the glottis and the trachea.

In other embodiments of the present disclosure, the extracted tracheal structure may not be displayed on the display so as not to distract the operator.

The extracted structure of the trachea 31 can be measured to determine the diameter of the trachea 31. As shown in FIG. 4, an ellipse or a circle 42 is fitted to an inner boundary of extracted structure of the trachea 31. Then, the ellipse/circle 42 is measured to determine the attributes of the trachea 31. For example, the major axis of the ellipse or the diameter of the circle correspond to the diameter of the trachea 31. Further, the radius of the trachea 31 can be determined as well. In some embodiments of the present disclosure, the diameter of the ellipse or the circle correspond to the diameter of the trachea 31. In some embodiments of the present disclosure, the area of the ellipse or the circle correspond to the area of the trachea 31.

The person skilled in the art will understand that other method for determining the attributes of the trachea 31 can be adopted as long as it can determine the attributes of the trachea 31 based on the extracted structure of the trachea 31. For example, the length of the gap between the two vocal cord 33 can be measured and the maxim length can be determined as the diameter of the trachea 31.

In some embodiments of the present disclosure, a representation of the at least one attribute of the trachea is displayed on a display of the video laryngoscope system. In some embodiments of the present disclosure, the representation of the attribute of the trachea can be displayed in a separate area that are dedicatedly assigned for the attribute. In other embodiments, the representation of the attribute of the trachea can be superimposed on the received image from the image acquisition device 20. As shown in FIG. 4, the ellipse/circle 42 used for determining the attributes of the trachea can be used and the graphical representation of the attributes and superimposed on the received image and displayed on the display 13, such that the operator may intuitively confirm whether the determined attributes of the trachea is correct. For example, if the displayed ellipse/circle is not appropriately located and/or sized, the operator may notice that the determined attributes of the trachea is not correct. Then, the operator may instruct the system 10 to correct the attributes of the trachea, for example, by moving the image acquisition device 20 and acquiring a new image of the glottis and the trachea. Further, even if the representation of the attributes of the trachea is not correct, for example, if the ellipse/circle is smaller or bigger than the trachea in the received image, the operator can estimate the correct attribute manually so as to save the time for instructing the system 10 to correct the attributes of the trachea.

In some embodiments of the present disclosure, in addition to the ellipse/circle, a double sided arrow or a line segment graphically representing the attribute (for example, the diameter) of the trachea can be displayed on the display 13.

In some embodiments of the present disclosure, as shown in FIG. 4, the numerical representation 43 of the attribute of the trachea, i.e., the calculated value of the attribute (for example, the diameter) of the trachea, can be displayed on the display 13. As shown in FIG. 4, the diameter of the trachea 31 is displayed on the right-bottom corner of the image. By displaying the calculated value of the attribute of the trachea on the display 13, the operator may read the calculated value while operating the laryngoscope system 10 without interrupting the operation.

In some embodiments of the present disclosure, the at least one attribute of the trachea is output by the speaker 17. By output the attribute of the trachea via the speaker, the operator may be informed of the attribution while operating the laryngoscope system 10 without interrupting the operation, and the people around the system 10 other than the operator may note the determined attribution as well.

In some embodiments of the present disclosure, a reference object with known size can be positioned near the glottis 32 and be captured by the image acquisition device 20. Then, in comparison with the reference object, the trachea can be quantitatively assessed to determine at least one attribute of the trachea. In some embodiments, the reference object is a physical objection inserted into the subject's mouth and positioned near the glottis 32. In some embodiments, the reference object is projected on the tissue of the subject, such as the laser dots having constant intervals therebetween.

In some embodiments of the present disclosure, the magnification of the lens of the image acquisition device 20 and the distance between lens of the image acquisition device 20 and the tracheal structure are required in order to determine the at least one attribute of the trachea. For the objects have the same lengths in the image capture by the image acquisition device 20, if the magnification of the lens and the distance between lens and objects are different, the objects may have different actual lengths.

In some embodiments of the present disclosure, the magnification of the lens of the image acquisition device 20 is determined and stored in the memory 11. Further, the focal length and the image distance of the image acquisition device 20 is also determined and stored in the memory 11, and thus the object distance can be determined as well. During the quantitative assessment of the trachea, the operator can place the glottis 32 (e.g., the vocal cord 33) such that the glottis 32 is in focus, and then the distance between the lens of the image acquisition device and the glottis 32 equals to the predetermined object distance. In this case, the attribute of the trachea 31 can be determined based on the received image in view of the magnification and the object length of the lens of the image acquisition device 20. In this embodiment, the calculation of the attribute of the trachea 31 is simple and the operation of the operator is convenient.

In some embodiments of the present disclosure, the video laryngoscope system 10 further includes a distance measuring device to measure the distance between lens of the image acquisition device 20 and the tracheal structure. For example, the distance measuring device may adopt any ranging technologies known in this technical filed, such as laser, phase difference, flight of time, and interfering ranging technologies.

In the embodiments of the present disclosure, the video laryngoscope system 10 may comprise any machine configured to perform processing and/or calculations, may be but is not limited to a work station, a server, a desktop computer, a laptop computer, a tablet computer, a personal data assistant, a smart phone, or any combination thereof. The one or more processor 12 may be any kinds of processors, and may comprise but are not limited to one or more general-purpose processors and/or one or more special-purpose processors (such as special processing chips). The processor 12 may include one or more application specific integrated circuits (ASICs), one or more general purpose processors, one or more controllers, one or more programmable circuits, or any combination thereof. Further, the memory 11 may be any storage devices that are non-transitory and can implement data stores, and may comprise but are not limited to a disk drive, an optical storage device, a solid-state storage, hard disk or any other magnetic medium, a compact disc or any other optical medium, a ROM (Read Only Memory), a RAM (Random Access Memory), a cache memory and/or any other memory chip or cartridge, and/or any other medium from which a computer may read data, instructions and/or code. The communications device 16 may be any kinds of device or system that can enable communication with external apparatuses and/or with a network, and may comprise but are not limited to a modem, a network card, an infrared communication device, a wireless communication device and/or a chipset such as a Bluetooth™ device, 1302.11 device, WiFi device, WiMax device, cellular communication facilities and/or the like.

Software elements may be located in the memory 11, including but are not limited to an operating system, one or more application programs, drivers and/or other data and codes. Instructions for performing the methods and steps described in the above may be comprised in the one or more application programs, and the parts of the aforementioned system 10 may be implemented by the processor 12 reading and executing the instructions of the one or more application programs. The executable codes or source codes of the instructions of the software elements may also be downloaded from a remote location.

It should also be appreciated that variations may be made in accordance with specific requirements. For example, customized hardware might also be used, and/or particular elements might be implemented in hardware, software, firmware, middleware, microcode, hardware description languages, or any combination thereof. For example, some or all of the disclosed methods and devices may be implemented by programming hardware (for example, a programmable logic circuitry including field-programmable gate arrays (FPGA) and/or programmable logic arrays (PLA)) with an assembler language or a hardware programming language (such as VERILOG, VHDL, C++) by using the logic and algorithm according to the present disclosure.

Those skilled in the art may clearly know from the above embodiments that the present disclosure may be implemented by software with necessary hardware, or by hardware, firmware and the like. Based on such understanding, the embodiments of the present disclosure may be embodied in part in a software form. The computer software may be stored in a readable storage medium such as a floppy disk, a hard disk, an optical disk or a flash memory of the computer. The computer software comprises a series of instructions to make the computer (e.g., a personal computer, a service station or a network terminal) execute the method or a part thereof according to respective embodiment of the present disclosure.

The steps of the method 100 presented above are intended to be illustrative. In some embodiments, method may be accomplished with one or more additional steps not described, and/or without one or more of the steps discussed. In some embodiments, method may be implemented in one or more processing devices (e.g., a digital processor, an analog processor, a digital circuit designed to process information, an analog circuit designed to process information, a state machine, and/or other mechanisms for electronically processing information). The one or more processing devices may include one or more modules executing some or all of the steps of method in response to instructions stored electronically on an electronic storage medium. The one or more processing modules may include one or more devices configured through hardware, firmware, and/or software to be specifically designed for execution of one or more of the steps of method.

Although aspects of the present disclosures have been described by far with reference to the drawings, the methods, systems, and devices described above are merely exemplary examples, and the scope of the present invention is not limited by these aspects, but is only defined by the appended claims and equivalents thereof. Various elements may be omitted or may be substituted by equivalent elements. In addition, the steps may be performed in an order different from what is described in the present disclosures. Furthermore, various elements may be combined in various manners. What is also important is that as the technology evolves, many of the elements described may be substituted by equivalent elements which emerge after the present disclosure.

1. A video laryngoscope system comprising:
an image acquisition device configured to capture images of glottis and trachea of a subject,
a memory configured to store one or more series of instructions,
one or more processor configured execute the series of computer instructions stored in the memory such that the video laryngoscope system performs the following steps:
receiving the images of the glottis and the trachea captured by the image acquisition device,
analyzing the received images to identify a tracheal structure, and
quantitatively assessing the trachea based on the identified tracheal structure, to determine at least one attribute of the trachea.

2. The system of claim 1, wherein image segmentation algorithm is applied to the captured images to identify the tracheal structure.

3. The system of claim 2, wherein the image segmentation algorithm comprises at least one of region growing algorithms, segmentation algorithms based on edge detection, segmentation algorithms based on neural network and segmentation algorithms based on machine learning.

4. The system of claim 1, wherein a representation of the identified tracheal structure is superimposed on the received image and displayed on a display of the video laryngoscope system.

5. The system of claim 1, wherein at least one attribute of the trachea comprises at least one of a diameter of the trachea, a radius of the trachea, a perimeter of the trachea, an area of the trachea.

6. The system of claim 1, wherein a representation of the at least one attribute of the trachea is displayed on a display of the video laryngoscope system.

7. The system of claim 6, wherein the representation of the at least one attribute of the trachea is superimposed on the received image.

8. The system of claim 6, wherein the representation of the at least one attribute of the trachea comprises graphical representation and numerical representation of the attribute of the trachea.

9. The system of claim 1, wherein the at least one attribute of the trachea is output by a speaker

10. The system of claim 1, wherein the image acquisition device has predetermined magnification and object distance, and is positioned such that the glottis is in focus.

11. The system of claim 1, wherein a reference object with known size is positioned near the glottis and is captured by the image acquisition device.

12. The system of claim 1, further comprising a distance measuring device configured to measure the distance between a lens of the image acquisition device and the tracheal structure.

13. A method for quantitatively assessing a trachea comprising:
receiving images of glottis and trachea of a subject captured by an image acquisition device of a video laryngoscope system,
analyzing the received images to identify a tracheal structure, and
quantitatively assessing the trachea based on the identified tracheal structure, to determine at least one attribute of the trachea.

14. The method of claim 13, wherein analyzing the received images to identify a tracheal structure comprises applying image segmentation algorithms to the captured images to identify the tracheal structure.

15. The method of claim 14, wherein the image segmentation algorithms comprise at least one of region growing algorithms, segmentation algorithms based on edge detection, segmentation algorithms based on neural network and segmentation algorithms based on machine learning.

16. The method of claim 13, further comprising superimposing a representation of the identified tracheal structure on the received image and displaying the superimposed image on a display of the video laryngoscope system.

17. The method of claim 13, wherein at least one attribute of the trachea comprises at least one of a diameter of the trachea, a radius of the trachea, a perimeter of the trachea, an area of the trachea.

18. The method of claim 13, further comprising displaying a representation of the at least one attribute of the trachea on a display of the video laryngoscope method.

19. The method of claim 18, further comprising superimposing the representation of the at least one attribute of the trachea on the received image.

20. The method of claim 18, wherein the representation of the at least one attribute of the trachea comprises graphical representation and numerical representation of the attribute of the trachea.

21-24. (canceled)
